(19) United States
(12) Patent Application Publication
(10) Pub. No.: US 2007/0254038 A1
(43) Pub. Date: Nov. 1, 2007

(54) Microspheroidal Controlled Release Of Biomolecules

(75) Inventors: Paul Ducheyne, Rosemont, PA (US); Shulamith Radin, Voorhees, NJ (US); Tiffany Lisa Chen, Hillsborough, CA (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: The Trustees of the University of Pennsylvania, Philadelphia, PA (US)

(21) Appl. No.: 11/739,962

(22) Filed: Apr. 25, 2007

Related U.S. Application Data
(60) Provisional application No. 60/795,608, filed on Apr. 26, 2006.

(51) Int. Cl. A61K 38/14; A61K 9/50; A61K 31/24

(52) U.S. Cl. 424/490; 514/8; 514/535

(57) ABSTRACT

Silica-based xerogel microspheres are provided containing pharmaceutically active compounds. These microspheres are robust, release active compounds at predictable rates and may provide such release for relatively long periods of time. Pharmaceutical compositions, methods for delivering medicaments and methods for treatment of disease states or conditions, inter alia, infection or pain, as well as methods for fabrication of such microspheres, are also provided.

### CROSS REFERENCE TO RELATED APPLICATION

This application claims priority to U.S. Provisional Application Ser. No. 60/795,608, filed Apr. 26, 2006, the entire disclosure of which is incorporated herein by reference in its entirety.

### FIELD OF THE INVENTION

The present invention is directed to the preparation of xerogel microspheres. The xerogel films of this invention contain a pharmaceutically active compound or compounds, which compounds may controllably released into body fluids or body tissues when the microspheres are placed in the body of a patient or into contact with body fluid.

### BACKGROUND OF THE INVENTION

Controlled release focuses on delivering biologically active agents locally over extended time periods (Heller, J., “Use of polymers in controlled release of active agents”, Controlled Drug Delivery: Fundamentals and Applications, Robinson, Jr, et al., editors, New York, Dekker, 1987; Radin, S, Ducheyne, P., “Nanostructural control of implantable xerogels for the controlled release of biomolecules”, Learning from Nature How to Design New Implantable Materials: from Biomineralization Fundamentals to Biomimetic Materials and Processing Routes, Reis, R. L., and Weiner, S, editors, New York, Kluwer, 2005). The site specificity of the delivery reduces the potential side effects that can be associated with general administration of drugs through oral or parenteral therapy (Radin, S., ibid.; Kortesuo, P. et al., J. Control. Release 2001; 76(3):227-238). Prevalent mechanisms for the delivery of biological agents by controlled release devices are either resorption of the drug carrier material or diffusion. The resorption of these devices may, however, cause an inflammatory tissue response which interferes with the treatment sought for with the biomolecules (Ibim, S. M., et al., Poly(anhydride-co-imides): In vivo biocompatibility in a rat model, Biomat., 1998; 19:941-951).

Room temperature processed silica-based sol-gel materials are resorbable and biocompatible materials. Their biocompatibility reduces the risk of inflammatory response typically caused through the resorption of other carrier materials by the body during or after the delivery of the pharmaceutically active or other biologically active molecules.

Sol-gels are known per se as are many of the overall chemistries which can be used to prepare them. A convenient work summarizing sol-gel technology is Brinker, et al., Sol-Gel Science—The Physics and Chemistry of Sol-Gel Processing, Academic Press, 1990. Chapter 13 of Brinker, et al., which chapter is specifically incorporated herein by reference, discusses the formation of certain kinds of films from sol-gels, typically, silica based films. Brinker et al. focused on the effects of various processing parameters such as the sol composition (water concentration, alcohol concentration and pH of the sols); the incorporation of biologically active compounds was never considered. Thus, those authors never appreciated the need to alter processing properties to incorporate desirable quantities of medicaments, factors and other desirable therapeutic compounds in microspheres for subsequent, controlled release—all while maintaining stability, uniformity of “active” distribution, or potency.

Previously, certain bulk sol-gel materials have been prepared for use in orthopaedics and in selected therapeutic regimes. In some cases, biologically active moieties, such as bone morphogenic protein, antibiotics and other species have been included in such bulk sol-gels. These materials have been proposed for use in the body of patients, e.g. for use in surgery such as spine and other orthopaedic surgery as well as for use in drug delivery intracorporeally. The preparation of sol-gels generally, as well as sol-gels having pharmaceutically active species in them has been disclosed in a number of U.S. patents, including several assigned to the assignee of this invention. These include U.S. Pat. Nos. 5,874,109; 5,849,331; 5,817,327; 5,861,176; 5,871,777; 5,591,453; 5,830,480; 5,964,807; and 6,569,442. Ducheyne, et al., U.S. application Ser. No. 11/403,335, assigned to the assignee of this invention is of particular note. Each of these is incorporated herein by reference in its entirety in order to set forth a number of ways of preparing sol-gels generally useful to the present invention, especially certain sol-gels having pharmaceutical or other biologically active molecules included within them. In most of these, bulk materials were produced by room temperature processes that included an acid-catalyzed hydrolysis of a silica precursor (tetramethyl orthosilicate (TMOS) or tetraethyl orthosilicate (TEOS)) to form a liquid sol followed by sol casting, gelation, aging, and drying. The biologically active compounds were mixed into the liquid sols and became encapsulated in the resulting solids shaped either as discs or granules. The compounds, which were incorporated this way, were released in a controlled manner and maintained their biological activity.

Kortesuo, et al. (Int. J. Pharm., 2000; 200(2):223-229) have disclosed a process for manufacturing spray dried controlled release sol-gel microparticles. This process included the formation of acid-catalyzed liquid sol with incorporated drugs and subsequent spray drying. The resulting particles have a low surface area of about 1 m2/g typical for dense (non-porous) materials, suggesting that the important controlled release properties of highly porous room temperature processed sol-gels were lost as a result of the spray drying process.

Peterson, et al. (Proceedings of the Society for Experimental Biology and Medicine, 1998; 218(4):365-369) have disclosed an encapsulation of pancreatic islets into emulsified sol-gel spheres. The process conditions employed favor encapsulation of more lipophilic materials, such as cells, but adversely affects the solubility of more hydrophilic compounds, such as pharmaceutically active molecules, which may lead to low loading of actives, non-uniform distribution in the sol-gel and resultant particles, or inconsistent controlled release properties.

There remains a great need for materials useful in surgery, in therapeutics, for the treatment of wounds and otherwise which effect the controlled release of pharmaceutically or biologically active molecules. It has long been desired to provide materials, e.g. which are bacteriostatic and can be used in emergent therapy for wounds. Other materials are desired for use in surgery, especially orthopedic surgery while still other uses involving such controlled release of medicaments will find immediate application in diverse therapeutic regimes.

### SUMMARY OF THE INVENTION

While the films or composites disclosed above referenced Ducheyne, et al. application, for example have predictable drug release characteristics, the inventors herein have now recognized that when these materials are granularized or powdered from bulk-formed material, they possess angular geometries. The sharp corners of these geometries may elicit more of an inflammatory response or other undesired effects. In addition, their short and sustained drug release characteristics or the particles' stability may be adversely affected by jagged edges on the particles formed during their formation from bulk composite .

Previous sol-gel technologies were inapplicable to the preparation of xerogel particles without sharp edges, giving rise to surface cracks, jagged edges, non-uniform composition or delivery, low active molecule loading, low surface area, small pore volume, or insufficient porosity, and the like, making them ineffective for the controlled release of pharmaceutically or biologically active molecules.

The inventors herein have now discovered processes for the manufacture of xerogel microspheres which provides many, if not all, of the beneficial properties found heretofore only in the bulk xerogel composites. These are formed with substantially smooth geometries. They are generally spheroidal.

### BRIEF DESCRIPTION OF THE DRAWINGS

FIG. 1 is an optical micrograph of emulsified acid-base catalyzed silica xerogel microspheres. These microspheres are 100-300 μm in diameter (60×).

FIG. 2 shows a size distribution of drug-free microspheres produced at various stirring speeds, as measured by sieving. The dimensions of the various fractions are indicated in μm.

FIG. 3 depicts cumulative vancomycin release (μg/ml) from microspheres (MS) as a function of immersion time in phosphate buffered saline (PBS), load, and water/TEOS molar ratio.

FIG. 4 depicts The cumulative vancomycin release (μg/ml) from microspheres (MS) or granules (G) as a function of immersion time in PBS.

FIG. 5 is a plot of the rate of release plot of the cumulative vancomycin release from microspheres and granules vs. the square root of time.

FIG. 6 is a plot of the cumulative bupivacaine release (μg/ml) from microspheres (MS) or granules (G).

### DETAILED DESCRIPTION OF ILLUSTRATIVE EMBODIMENTS

The invention is directed, in part, to silica-based xerogel microspheres, comprising substantially spheroidal silica-based xerogel beads having a surface area of from about 100 to about 1000 m²/g; an average pore size of from about 1 to about 10 nm; and substantially within the beads, at least one biologically active compound, said biologically active compound being acid stable and soluble in water or water-compatible solvent in an amount of at least about 10 gm/l.

The present invention is also directed, in part, to controlled-release carriers having a generally spherical or spheroidal shape, at least by typical visual observation. In the carriers according to the invention, biologically active molecules are incorporated, or perhaps encased, within the matrix of a silica-based microsphere. We have found that a derivation of the sol-gel technique facilitates such incorporation without negatively affecting subsequent activity of the molecules. In the case of substantially pure silica microspheres, the release of the biological molecules from the carrier is effected primarily by diffusion through the pore structure. In the instance the microsphere contains oxides in addition to silicon, the release of biological molecules is effected by diffusion and reaction when immersed in fluids such as, for example, body fluids.

Typically, the microspheres of the present invention are substantially spheroidal in shape. By this it is meant that the microspheres are substantially free of any jagged edges, which may be formed by grinding, crushing or the like as previously practiced in the prior art. The microspheres may be described as being round, egg-shaped, or even potato-shaped bodies, or the like, so long as they are substantially free of any jagged edges, they are considered to be within the ambit of the present invention. Typically the diameter of the claimed microsphere will be in the range of about 1 to about 710 micrometers. By diameter, we mean, more broadly, the distance from a point on the side wall, through the center of the microsphere to the point opposite on the microsphere surface. Preferably the diameter, will be from about 105 to 710, more preferably 210 to 710, still more preferably 210-350 micrometers or any combination thereof. In general, the microspheres will comprise spheres or spheroidal shapes of any number of sizes within the diameter range herein noted, and the particular preferred range may depend upon the application or method chosen for the delivery of the biologically active compound. In certain preferred embodiments, the microspheres are spherical in nature.

It will be obvious to one or ordinary skill in the art that the surface area of the microsphere is not critical, provided that the surface is free of defects and/or jagged edges. Typically, the surface area will be in the range of from about 100 to about 1000 m²/g, preferably from about 200 to about 1000 m²/g, with from about 400 to about 1000 m²/g being more preferred.

Likewise, one of skill in the art will recognized the impact of average pore size and its advantages. Typically, the average pore size will be from about 1 to about 10 nm, preferably from about 2 to about 10 nm, with from about 2 to about 5 nm being particularly preferred.

In certain embodiments, the silica-based xerogel microspheres contain at least one biologically active compound. The biologically active molecules to be incorporated are added at concentrations resulting in final concentrations ranging from about 0.0001 to about 10% by weight of the microsphere.

As used herein, “biologically active compound” are defined as an organic molecule having an effect in a biological system, whether such system is in vitro, in vivo, or in situ.

In certain other embodiments of the silica-based xerogel microsphere, the biologically active compound is antibiotic, antineoplastic, antiangiogenic, antithrombogenic, anti-inflammatory, analgesic, a cytokine or a tissue growth stimulating moiety, growth factors, preferably bone growth factors. The compound may be prepared by any means known in the art, including, for example, organic synthesis or genetic engineering techniques. Non-limiting examples of useful biologically active compounds in the present invention are genetically engineered BMP-2, vancomycin, bupivacaine, or another analgesic. In certain more preferred embodiments, the compound is vancomycin. In other alternative preferred embodiments, the compound is bupivacaine or another analgesic.

The term “antibiotic” includes bactericidal, fungicidal, and infection-preventing drugs which are substantially water-soluble such as, for example, gentamicin, vancomycin, penicillin, and cephalosporins.

The term “type” as used hereinafter in reference to biologically active compounds refers to biologically active molecules of the previously listed categories, as well as specific compounds, i.e. vancomycin, TGF-beta, etc. These specific compounds can be in the same or different categories. It is also contemplated that two or more types of biologically active molecules can be contained in each microsphere or microsphere composition as defined herein. This can be effected by simultaneous addition of the molecules into the solution.

Since the biologically active compounds to be incorporated retain their biological activities after treatment in moderate to highly acidic conditions, an amount of acid necessary to maintain acidity in a range of pH from about 1-4.5, preferably about 1.5-3, prior to, or during, incorporation of biologically active molecules is used.

There are any number of ways to prepare silica-based xerogel microspheres, as noted in the specification and the references cited herein, each of which is incorporated herein by reference in its entirety. However, a preferred method of preparing the silica-based xerogel microsphere of the invention is by an emulsification process, particularly when the process utilizes a biocompatible liquid as a non-compatible emulsification phase.

As used herein, “silica-based” refers to the inclusion of a silicon oxide in the composition of the glass. Other oxides may also be present.

The silica-based xerogel microspheres may be prepared by any known means, but preferably are prepared from at least one silicon alkoxide. The alkoxide is not critical, although it is preferably derived from an alcohol that is, in part, and preferably substantially soluble in water, such as for example, methanol, ethanol, propanol, isopropanol, alkoxyethanol, and the like.

In certain embodiments, the silica-based xerogel microspheres are formed from silicon alkoxide in a medium miscible with water, more preferably from a liquid sol that is at least partially formed at acid pH. As a consequence, it is preferred that the biologically active compound is substantially stable at acid pH, that is, that contact with acid under the conditions of sol, xerogel, and/or microsphere formation does not substantially affect the structure and/or efficacy of the biologically active compound. From a more practical standpoint, the compound is considered acid stable if, after formation of the microsphere, the “active” meets standards for pharmaceutically acceptable shelf life. Alternatively, a compound is substantially soluble if it retains at least 50%, preferably 60%, more preferably 75%, still more preferably 90%, with at least 95% of its activity after formation of the microsphere.

Thus, the invention is directed in part to processes for preparing a silica-based xerogel microsphere, comprising treating a silicon alkoxide with acid to provide a sol; optionally adding water or water-compatible solvent to the sol; contacting the sol with biologically active compound substantially stable in the sol, preferably in the form of an aqueous or water miscible solution of the compound, to provide an essentially one-phase mixture; increasing the pH of the mixture; and emulsifying the mixture in a pharmaceutically acceptable, immiscible phase to yield the microsphere.

The order of addition of silicon alkoxide, acid, and water is not critical. Typically, one may add water to the acid-silicon alkoxide mixture. In certain preferred embodiments, water is added to the sol. In other embodiments, the acid maybe take a more dilute form initially. Once the acid-silicon alkoxide sol, with or without added water, is prepared, it may be contacted with at least one biologically active compound substantially stable in the sol, preferably to provide an essentially one-phase mixture. In some other preferred embodiments, two or more biologically active compounds are added to the sol. In some embodiments, the acid will take the form of an aqueous solution.

The level of acid is not critical to the formation of the sol, but may, if too high affect the stability of the biologically active compound. As general guidance, the acid level should be adjusted below that where the instability of the active becomes a major factor. Typically, the pH should be in the range of from about 0 to about 4, more preferably from about 1 to about 4, after the silicon alkoxide, acid, optional added water, and biologically active compound are brought together.

In certain preferred embodiments, the total water to silicon alkoxide molar ratio in the sol is in the range of from about 5 to about 20, and all combinations and subcombinations thereof. By total water, it is meant to include any water present in the sol after the silicon alkoxide, acid, optional added water, and biologically active compound are brought together. Typically the biologically active compound is dissolved in water or a water miscible solvent. The concentration of the compound in the sol is generally in the range of from about 5 mg to about 500 mg of biologically active compound per gram of SiO₂ contained in the sol. Typical non-limiting loadings of vancomycin are in the range of from about 10 to 30 mg per gram of SiO₂, preferably 20 to 30 mg per gram of SiO₂, contained in the sol. With bupivacaine, typical non-limiting loadings were in the range of from about 20 to 80 mg per gram of SiO₂, preferably 50 to 80 mg per gram of SiO₂, contained in the sol.

Stirring of the immiscible phase during the emulsification process is important, at least in that the speed of stirring affects the diameter of the microsphere formed. In general, stirring speeds of from about 220 to about 440 were adequate for formation of the microspheres, although slower or faster speeds could be utilized, especially where the gelation rate was outside the standard rate. Increasing the stirring speed led to a relatively greater distribution of smaller diameter microspheres within the general range of expected size microspheres as well as extending the lower diameter range of microspheres prepared. Slower speeds analogously gave relatively greater distributions of larger diameter microspheres.

Once the essentially one-phase mixture of sol and biologically active compound or compounds is formed, the pH is increased by the addition of base. In some embodiments, the base is water soluble or soluble in a water-miscible solvent, preferably water. In preferred embodiments, the base is ammonium hydroxide.

Base is added to decrease the time to gelation. Although the amount of base added may vary, it is important that the subsequent emulsification be carried out prior to gelation. Therefore the more base added, the more quickly the sol must be emulsified to provide the microspheres of the invention. As a rule of thumb, the amount of base added should bring the pH of the sol to between about 4 and about 6, preferably 4.5 to 6, with about 5.5 being preferred. The addition of base should reduce the gelation time to between about 5 minutes and about 4 hours, preferably about 5 minutes and about 2 hours, more preferably about 5 minutes and about 1 hour, with about 15 to about 30 minutes being even more preferred.

Once the base has been added, but before gelation, the now base-treated sol incorporating biologically active compound is emulsified by addition to a water-immiscible phase, preferably wherein the immiscible phase is biocompatible. Typically, the volume/volume ratio of sol to oil during emulsification was about 5/100 to about 10/100. Optimization of other parameters, such as for example, drip rate or droplet size, temperature and or viscosity of the oil phase are among the parameters that would be obvious to one skilled in the art, once armed with the present invention.

The invention is also directed, in part to, pharmaceutical compositions, comprising a pharmaceutically acceptable carrier; and at least one silica-based xerogel microsphere as described herein.

Further embodiments of the invention include methods for delivering a medicament to a patient in need thereof, comprising the step of administering to said patient an effective amount of at least one silica-based xerogel microsphere as described herein, preferably wherein the medicament administered through use of a silica-based xerogel microsphere as described herein comprises vancomycin or bupivacaine.

Among other embodiments, the present invention is directed to methods for treating a disease state or condition in a patient in need thereof, comprising the step of administering to said patient an effective amount of at least one silica-based xerogel microsphere as described herein, preferably wherein the disease state or condition treated is infection or pain.

### EXAMPLES AND EXPERIMENTAL METHODS

Sol-gel derived silica microspheres were synthesized using acid-base catalyzed hydrolysis of tetraethoxysilane (TEOS, Strem Chemicals, Newburyport, Mass.) followed by emulsification. An acid-base catalysis sequence was selected rather than an acid catalysis in order to shorten the time to gelation of the sol. A shorter time to gelation is preferred for the production of sol-gel microspheres by emulsification.

Typical Sol Synthesis

TEOS (10 ml) and 0.1 M HCl (2.4 ml), with or without the addition of deionized water, were mixed using a magnetic stirrer until a one-phase sol was formed. The water/TEOS molar ratio varied from 0 to 10. Pharmaceutical agents were then added to the sol. For example, Sols with 20 mg/g and 30 mg/g of vancomycin (drug to SiO2 ratio), and sols with 50 mg/g and 80 mg/g of bupivacaine per gram SiO₂ were made by adding corresponding amounts of the drug. The sol containing added pharmaceutical agents was mixed for 30 minutes at 660 rpm and was then allowed to stand for 15 minutes. Subsequently, 0.08 M NH4OH (2.2-2.4 ml) was added dropwise to the sol, which was stirred at 660 rpm targeting a final pH of about 5.5. Under these conditions, the time to gelation varied from about 20 and 40 minutes. Upon mixing, the sol was added dropwise into vegetable oil stirred at speeds between 220 and 440 rpm and microspheres precipitated to the bottom of the beaker. Microspheres were filtered through a 70 μm nylon microporous filter and then rinsed with DI water and alcohol. The microspheres were left to dry overnight in a laminar flow hood.

Addition of Biologically Active Compounds—Variation of the Water/Alkoxysilane Molar Ration (R)

Vancomycin (vancomycin-HCl; Abbott Labs, Chicago, Ill.) dissolved in water at 100 mg/ml was used for incorporation into the sols. Bupivacaine (Spectrum, New Brunswick, N.J.) dissolved in methanol at 70 mg/ml was also used for incorporation into the sols.

Certain water amounts were found to be preferred because they led to a clear sol without precipitation of the biologically active compounds. The effect of water content on the incorporation of vancomycin into the sol was studied by using either “water-free” (no additional water added other than that contained in the aqueous HCl solution) acid-catalyzed sols or sols with added water/TEOS molar ratios (R) of 5, 6, 8, and 10. As shown in Table 1, “water-free” acid-catalyzed sols became cloudy upon drug addition, indicating precipitation of the drug. When water was added at R=4, low doses of vancomycin such as 16.7 mg/g SiO₂ could be added to the acid-catalyzed sol. However, precipitation of vancomycin was seen when base was added. When water was added to achieve R=5, low doses (doses up to 20 mg/g SiO₂) were successfully incorporated in the sol-gel. That is, no precipitation was observed after the addition of the drug and base. At some higher dose levels, vancomycin precipitation was observed (such as 28 mg/g SiO₂) after incorporation of the base. By use of higher water/TEOS ratios (8 and above) were the higher loads (such as 28 mg/g SiO₂) successfully incorporated. This suggests that, in contrast to the water-free sol-gel synthesis of microspheres as described by others, incorporation of these drugs requires the presence of additional water and R values greater than 5.

TABLE 1
The effects of water/TEOS molar ratios (R) and vancomycin load (drug
to SiO₂ ratio in weight %) on the incorporation of vancomycin into
acid-catalyzed (AC) and acid-base catalyzed (ABC) sols.

| | | Water to TEOS molar ratio (R) | | | | | | | | |
|---|---|---|---|---|---|---|---|---|---|---|
| Vancomycin | | Water-free | 4 | | 5 | | 8 | | 10 | |
| loading | | AC sol | AC | ABC | AC | ABC | AC | ABC | AC | ABC |
| 16.7 | mg/g | cloudy | clear | cloudy | clear | clear | — | — | — | — |
| 22.2 | mg/g | — | — | — | clear | clear | clear | clear | clear | clear |
| 28 | mg/g | — | — | — | clear | cloudy | — | — | — | — |
| 33 | mg/g | — | — | — | — | — | clear | clear | — | — |

The addition of pharmaceutical agents and the variation in R also altered the pH and time to gelation of the sol. The volume of base was modified to maintain the time to gelation within the preferred range of 20 to 40 minutes.

Materials Characterization

Morphology and size distribution of the microspheres were determined microscopically using an image analysis system (Image-Pro Plus 4.0). Sieving was also used to determine the size distribution. Nylon microporous filters of 70, 105, 210, 350, 500, and 710 μm were used to separate the microspheres. Surface area and average pore size may be determined using B.E.T. analysis.

In Vitro Release Kinetics

Acid-base catalyzed sols with incorporated drugs were also used to produce sol-gel granules via casting. 1 ml of acid-base catalyzed sols was cast into vials, aged for 3 days and dried at room temperature until there was no further weight-loss. The resulting sol-gel discs were crushed and then sieved to produce granules in the size range from 210 to 500 μm.

In vitro release was studied in phosphate buffered saline (PBS, Gibco, pH=7.4) at 370 C with daily solution exchange using microspheres and granules between 210-500 μm. 5 mg of sol-gel particles were immersed in 1 ml of solution.

The concentration of the drug released was measured every 24 hours. Vancomycin and bupivacaine standards were prepared by dissolving appropriate amounts of the drug in PBS. Bupivacaine was dissolved in PBS through gradual heating in a water bath to 55° C. The release of vancomycin and bupivacaine was measured spectrophotometrically at 280 and 265 nm respectively.

Microsphere Characterization

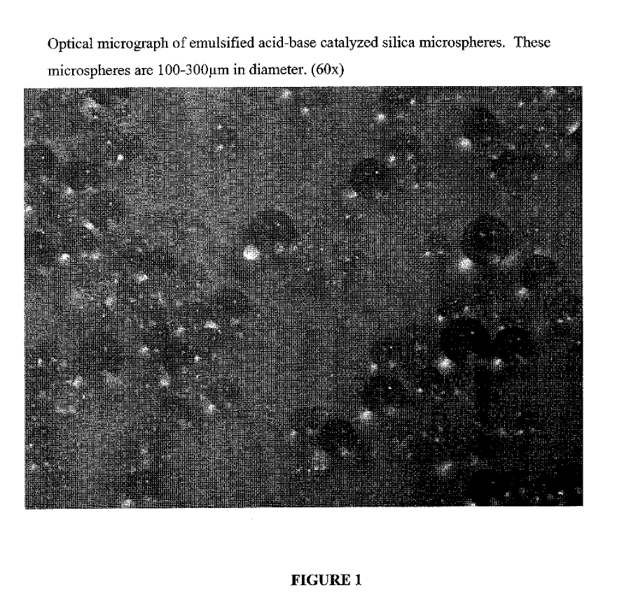

After addition of the base and prior to gelation of the sol, the sol was added dropwise to an non-water miscible phase such as vegetable oil stirred at a rate, typically in the range of about 220 to about 440 rpm. The emulsified silica-based xerogel precipitated as microspheres, with and without incorporated pharmaceutically active materials, which were removed by simple filtration using the appropriately pore-sized filter. Both types of microspheres, either drug-free or drug-containing, had ideally smooth, defect-free surfaces (FIG. 1).

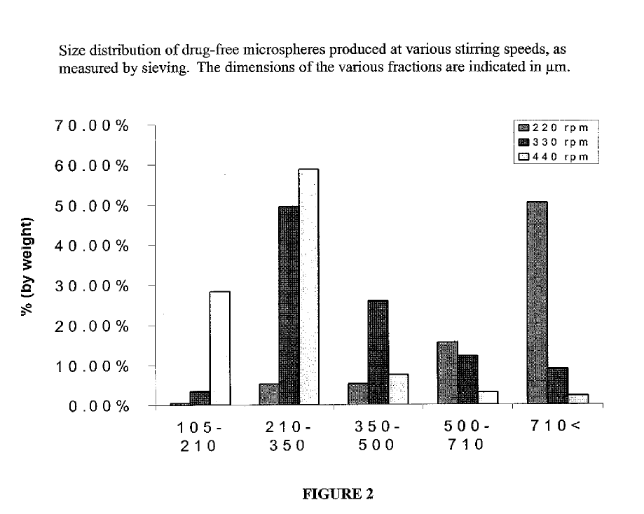

We found that the size of the microspheres was mainly dependent on the speed of stirring during emulsification. The size distribution as a function of speed of stirring is shown in FIG. 2. Lower speeds around 220 rpm about 50% of microspheres formed were greater than 710 μm, and non-spherical amorphous chunks precipitated along with the microspheres. When the speed of stirring increased, the size of microspheres decreased. At 330 rpm, about 50% of the microspheres were in the size range of 210 to 350 μm. At 440 rpm, the percentage of the microspheres in the size range of210 to 350 μm was increased to almost 60%. The percentage of the microspheres in the size range of 105 to 210 μm also was substantially increased to about 28% from less than 4% at the emulsification speed of 330 rpm.

Release Study of Vancomycin and Bupivacaine from Microspheres and Granules

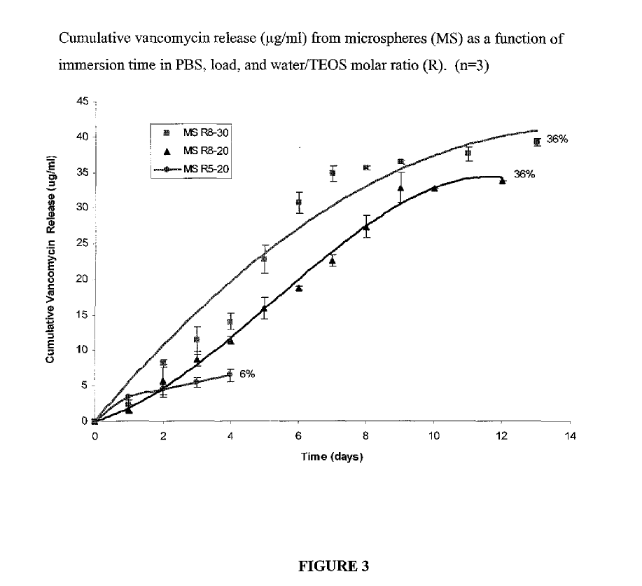

The cumulative release of vancomycin as a function of elution time, load, and water/TEOS molar ratio (R) is demonstrated in FIG. 3. It was found that microspheres with theoretical vancomycin concentrations of 20 mg/g, which were synthesized by using the water/TEOS ratio of 5, released only 6% of the original load. With increase of the (R) ratio up to 8, the rate of release and the amount released significantly increased: 36% of the original load was released over 12 days. At this theoretical load, the microspheres with R=8 and R=5 had a total release of 33.8 μg/ml and 6.5 μg/ml of vancomycin, respectively. When the load in the R=8 microspheres was increased up to 30 mg/g, the further increase of the rate of release and the total amount released was observed. These microspheres synthesized with water/TEOS of 8 showed time-and-load-dependent release.

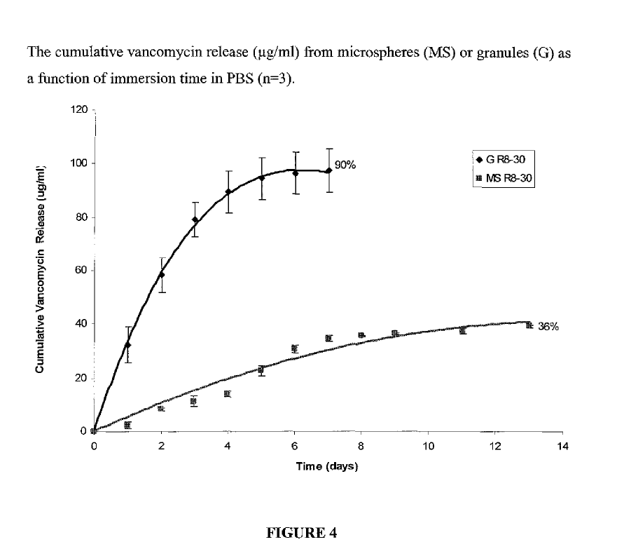

The data in FIG. 4 also demonstrates a dramatic difference in the release profiles from microspheres and granules derived from similarly synthesized sols (R=8, 30 mg/g of vancomycin load). In comparison to a fast and short term release from granules, vancomycin release from microspheres shows a slower and longer release. In addition, a higher percentage of the original vancomycin load was released from sol-gel granules. In the first three days, the sol-gel granules released about 80% of the load within of the elution study while the microspheres only released 7.5%. The granules released a total of 90% of the load over seven days and the microspheres released 36% of the total load over 14 days.

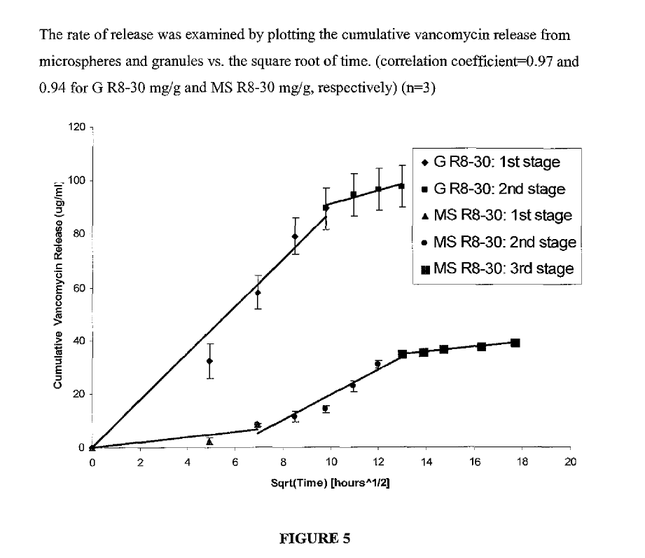

The stages of the release from granules and microspheres (R=8, 30 mg/g) were analyzed by plotting the release data against the square root of time (FIG. 5). These plots showed that the release profile of the sol-gel granules has two stages. In the first stage, the sol-gel granules demonstrated a fast, first-order release for the first 3 days of the elution study. This was followed by a slower, steady release for the final four days of the study. The first order release suggests a diffusion controlled mechanism. In comparison to granules, microspheres showed three stages of release: the first stage of delayed release over two days, the second stage of a faster, first-order release over five days, and the third stage of a slower release.

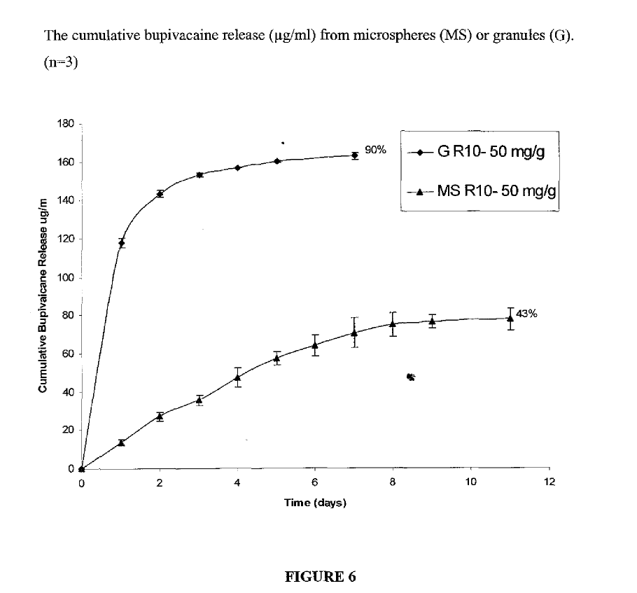

As shown in FIG. 6, microspheres with incorporated bupivacaine also showed a time dependent long-term release. Similarly to incorporated vancomycin, release profiles of bupivacaine from microspheres and granules were remarkably different. In the case of granules, a burst release of 80% of the load on day 1 and 90% release over 6 days were observed. In contrast, microspheres demonstrated a more gradual release over longer period of time: 43% of the original load was released over 10 days. The analysis of the release data plotted against the square root of time (not shown) indicated that, similar to vancomycin release, microspheres with bupivacaine also demonstrated a three stage release with a first stage of delayed release, followed by a second stage of a faster release of 1st order, and, subsequently, a third stage of a slower release. In contrast, the granules did not show any delay. A two stage release with a first stage of a fast release of 1st order release followed by a 2nd stage of a steady and slower release was observed.

What is claimed:

1. Silica-based xerogel microspheres, comprising:
substantially spheroidal silica-based xerogel beads having
a surface area of from about 100 to about 1000 m²/g; and
an average pore size of from about 1 to about 10 nm; and
substantially within the bead, at least one biologically active compound, said compound being acid stable and soluble in water or water-compatible solvent in an amount of at least about 10 gm/l.

2. The silica-based xerogel microspheres according to claim 1, wherein the silica-based xerogel microspheres are formed from silicon alkoxide.

3. The silica-based xerogel microspheres according to claim 1, wherein the silica-based xerogel microspheres are formed from silicon alkoxide in a medium miscible with water.

4. The silica-based xerogel microspheres according to claim 1, wherein the silica-based xerogel microspheres result from a liquid sol at least partially formed at acid pH.

5. The silica-based xerogel microspheres according to claim 1, wherein biologically active compound is antibiotic, antineoplastic, antiangiogenic, antithrombogenic, anti-inflammatory, analgesic, a cytokine or a tissue growth stimulating moiety.

6. The silica-based xerogel microspheres according to claim 1, wherein the biologically active compound comprises vancomycin.

7. The silica-based xerogel microspheres according to claim 1, wherein the biologically active compound comprises bupivacaine or another analgesic.

8. The silica-based xerogel microspheres according to claim 1, wherein the surface area is from about 200 to about 1000 m²/g.

9. The silica-based xerogel microspheres according to claim 8, wherein the surface area is from about 400 to about 1000 m²/g.

10. The silica-based xerogel microspheres according to claim 1, wherein the average pore size is from about 2 to about 10 nm.

11. The silica-based xerogel microspheres according to claim 10, wherein the average pore size is from about 2 to about 5 nm.

12. The silica-based xerogel microspheres according to claim 1, wherein the microspheres are formed by an emulsification process.

13. The silica-based xerogel microspheres according to claim 12, wherein the emulsification uses a biocompatible liquid as a non-compatible emulsification phase.

14. The silica-based xerogel microspheres according to claim 1, having a diameter in the range of about 1 to about 710 micrometers.

15. A process for preparing silica-based xerogel microspheres, comprising:
treating a silicon alkoxide with acid to provide a sol;
optionally adding water or water-compatible solvent to the sol;
contacting the sol with biologically active compound substantially stable in the sol to provide an essentially one-phase mixture;
Increasing the pH of the mixture; and
emulsifying the mixture in a pharmaceutically acceptable, immiscible phase to yield the microspheres.

16. The process of claim 15, wherein the pH increase reduces the gelation time of the mixture to between about 5 and about 60 minutes.

17. The process of claim 16, wherein the pH increase reduces the gelation time of the mixture to between about 15 and about 30 minutes.

18. The process according to claim 15, wherein water is added to provide the one-phase mixture.

19. The process according to claim 15, wherein the base is ammonium hydroxide.

20. The process according to claim 15, wherein the immiscible phase is biocompatible.

21. A pharmaceutical composition, comprising a pharmaceutically acceptable carrier; and microspheres according to claim 1.

22. A method for delivering a medicament to a patient in need thereof, comprising the step of administering to said patient an effective amount of microspheres according to claim 1.

23. The method of claim 22, wherein the medicament comprises vancomycin or bupivacaine.

24. A method for treating a disease state or condition in a patient in need thereof, comprising the step of administering to said patient an effective amount of microspheres according to claim 1.

25. The method of claim 24, wherein the disease state or condition is infection or pain.
